(19) United States
(12) Patent Application Publication
(10) Pub. No.: US 2010/0100009 A1
(43) Pub. Date: Apr. 22, 2010

(54) Device Having a Hydrophilic Coating Comprising P-Toluene-Sulfonamide and a Method for the Preparation Thereof

(76) Inventors: Bo Rud Nielsen, Alleroed (DK); Niels Joergen Madsen, Alleroed (DK)

Correspondence Address: JACOBSON HOLMAN PLLC, 400 SEVENTH STREET N.W., SUITE 600, WASHINGTON, DC, 20004, US

(21) Appl. No.: 11/794,533

(22) PCT Filed: Dec. 23, 2005
(86) PCT No.: PCT/DK05/00830

§ 371 Date: Dec. 8, 2009

(30) Foreign Application Priority Data
Dec. 30, 2004 (DK) PA 2004 02030

(51) Int. Cl. A61L 29/08; B05D 3/02; C08K 5/435; A61M 25/00; A61M 25/09; A61L 31/10

(52) U.S. Cl. 600/585; 427/393.5; 524/169; 604/523

(57) ABSTRACT

The present invention provides a medical device having a substrate polymer surface carrying thereon a hydrophilic coating comprising a cross-linked hydrophilic polymer and p-toluenesulfonamide, and a method for the preparation thereof.

### FIELD OF THE INVENTION

The present invention relates to a device, suitably a medical device, carrying a hydrophilic coating comprising a cross-linked hydrophilic polymer and p-toluenesulfonamide, which has a low friction when wet. The invention relates to a method for applying such a hydrophilic coating on a substrate polymer surface of a device, devices obtainable by said method as well as a polymer coating solution containing p-toluenesulfonamide.

The hydrophilic coating according to the invention may be used for coating the surface or a part of a surface of a wide range of products in order to provide low friction properties to the surface. As examples of products which may be provided with a surface having a low friction when wet are medical instruments and devices such as catheters, endoscopes and laryngoscopes, tubes for feeding or drainage or endotracheal use, guide wires, condoms, barrier coatings (e.g. for gloves), wound dressings, contact lenses, implants, extracorporeal blood conduits, membranes (e.g. for dialysis), blood filters, devices for circulatory assistance or non-medical products such as packaging for foodstuff, razor blades, fishermen's net, conduits for wiring, water pipes having a coating inside, water slides, sports articles, cosmetic additives, mould release agents, and fishing lines and nets.

### BACKGROUND OF THE INVENTION

The application of hydrophilic coatings on devices, suitably medical devices, has become a very important method for improving biocompatibility between living tissue and the device.

Medical devices like catheters, guide wires, endoscopes etc. are often sliding in direct contact with the surface of living tissue when in use. Catheters and guide wires may e.g. be introduced into the blood vessels. Catheters for draining urine are typically introduced through natural (the urethra) or artificial body openings. Catheters may be withdrawn immediately after emptying the bladder or after some time when performing more or less permanent catheterisation. In both applications, the medical device is sliding in direct contact with a physiological surface, the walls of the blood vessels, and the mucosa of the urethra, respectively.

An important property of hydrophilic coatings that they reduce the friction and render biomedical devices slippery when wet and thereby reduce or avoid discomfort to the patient as well as any physiological damage and degeneration which may be caused by the medical device. A high soft abrasion resistance (low soft abrasion loss) of the coating is an advantageous property preventing loss of polymer coating when a nurse or doctor manipulates the device, e.g. a guide wire. Furthermore, a high hard abrasion resistance (low hard abrasion loss) is an advantage when the device comes into contact with hard surfaces. e.g. when a guide wire is removed from a plastic dispenser.

Hydrophilic coatings having a low friction coefficient when wet typically comprises hydrophilic polymers such as polyvinyl pyrrolidone (PVP), polycarboxylic acids, esters, salts and amides of poly(meth)acrylic acid, copolymers of poly(methyl vinyl ether/maleic anhydride) and polyglycols like polyethyleneglycol (PEG).

Such hydrophilic coatings are highly lubricious when wet as the coatings take up a significant amount of water, which leaves a non-bonded layer of free water molecules at the surface of the coating. The non-bonding character of the surface water is believed to cause the low friction of the wet coating. Hence, the use of such coatings on a biomedical or other device will improve biocompatibility and patient compliance. However, for most applications there will be high demands to the internal bonding strength of the coating.

According to Y. Fan (In Fan Y. L. 1990: “Hydrophilic Lubricious Coatings for Medical Applications”, Amer. Chem., Polym. Mater. Sci. Eng., 63:709-716.), the methods described in the literature by which hydrophilic coatings can be applied onto a substrate can roughly be divided into 5 different methods:

(1) Simple coating with hydrophilic polymers,

(2) Blending or complexing of hydrophilic polymers,

(3) Formation of interpenetrating polymeric networks,

(4) Coating with chemically reactive hydrophilic polymers and

(5) Surface grafting of hydrophilic monomers.

Hydrophilic coatings prepared by the first three methods generally have low abrasion resistance giving the devices a short effective lifetime. A considerable amount of polymeric residuals is released where the coated device comes into contact with other surfaces, e.g. at the site where it is introduced, and at the same time, this loss of polymeric material rapidly increases the friction coefficient. The abrasion or dissolution may be so pronounced that the reduction of the friction is not effective during the service period of the medical device and the low friction may even have vanished when the device is to be retracted.

The fourth method involves the use of chemically reactive hydrophilic polymers that are chemically bonded to substrates or primers containing e.g. aldehyde, epoxy or isocyanate groups. As an example, U.S. Pat. No. 4,373,009 discloses that a hydrophilic layer may be formed on a substrate, e.g. wound drains, catheters, surgical tools and arteriovenous shunts, by binding unreacted isocyanate groups on the substrate surface and treating the surface with a hydrophilic copolymer made from vinyl-pyrrolidone monomers and monomers containing an active hydrogen adapted to form covalent bonds with the isocyanate. This coating method suffers from the drawback of the use of toxic, reactive materials and in order to avoid a residual toxic effect there is a demand of long reaction times and eventually washing steps in the process.

The fifth method typically involves the formation of radicals on the surface of isolated chains of saturated substrate polymer, such as polyurethane. The radicals may be formed e.g. directly by UV-irradiation of the system, indirectly by UV-irradiation of the system with a small amount of photoinitiator capable of forming reactive radicals, directly by electron beam-irradiation of the system, or directly by gamma-irradiation of the system. The surface radicals then initiate the polymerisation of the hydrophilic monomer (e.g. acrylamide), which forms the hydrophilic coating. This method suffers from the drawback that some residual monomer will remain in the product, and this has to be removed in a separate purification step.

The method describe in e.g. WO 2004/0569090A1 differs from the five other methods and typically involves the formation of radicals on the surface of isolated chains of saturated substrate polymer, such as polyurethane, and on isolated chains of saturated hydrophilic polymer, such as PVP, which has been coated onto the substrate e.g. by dip coating. The radicals may be formed e.g. directly by UV-irradiation of the system, indirectly by UV-irradiation of the system with a small amount of photoinitiator capable of forming reactive radicals, directly by electron beam-irradiation of the system, or directly by gamma-irradiation of the system. By the subsequent combination of the radicals covalent bonds are formed (i) between the substrate and the hydrophilic polymer, and (ii) between the isolated chains of the hydrophilic polymer. Optimally, this results in a slightly crosslinked hydrophilic coating, which has high abrasion stability, good water binding capacity and low friction when it is wet. A further advantage of this method is that no monomers are involved, and if oligomeric or polymeric photoinitiators are used, the amount of extractables may be kept very low. However, this method requires UV-equipment or expensive electron beam- or gamma-irradiation equipment.

Thus, there are a number of ways to provide hydrophilic coatings on medical devices either based on coating with two layer systems where the first layer serves as a base layer or by coating with single layer system where covalent bonding to the substrate and polymer cross-linking are used for achieving coating strength.

However, there is still a need for alternative or even improved stable and lubricious coatings for devices, in particular medical devices.

It has now surprisingly been found that coatings with lower friction and high soft and hard abrasion resistance may be prepared by incorporating p-toluenesulfonamide into a coating comprising a cross-linked hydrophilic polymer.

Furthermore, it has been found that the coating of the invention has a lower tendency to stick or adhere to the biological tissue or to a polymer surface when used for coating of guide wires or other medical devices, which during use slides against biological tissue or polymer surfaces. This also applies to the initial phase where the surfaces come into contact with each other or are in contact with each other but have not yet started sliding against each other. The introduction of a guide wire not coated according to the invention through a tube of a plastic material may be difficult. The low tendency to exhibit what is known as the slip-stick phenomenon means that a medical device with a coating of the invention has a reduced adherence to biological tissue and initially to an introducer tube when the surfaces start sliding against each other.

Moreover, the coating of the invention may easily be applied to the substrate polymer surface, by simply dipping the polymer surface in a coating solution, followed by drying and curing.

European Patent application 0 514 913 A2 discloses the use of alkylated benzenesulfonamides or o/p-toluenesulfonamide together with a specific swelling agent in a 1:1 mixture followed by extensive washing in order to plasticize medical catheters made of polyamide or polyurethane. However, this treatment is very different from a coating procedure, and no mention is made of any reduced friction or increased abrasion resistance of the resulting catheters, since the sole purpose of the treatment is to make the materials softer in a fast and reliable way.

UK Patent application 2 048 897 describes the use of p-toluenesulfonamide in a primer coat on thermoplastic rubbers, which improves the adhesion between the thermoplastic rubber and polar surfaces such as urethane polymers. However, the scope of the present invention is quite the opposite, i.e. to decrease the adhesion and increase the abrasion resistance of a hydrophilic coating.

U.S. Pat. No. 4,260,531 discloses the use of p-toluenesulfonamide as plasticizer in an ink based on a styrene-acrylic copolymer resin for ink jet printing on polyolefins, which improves the adhesion to the polyolefin. However, no mention is made of any reduced friction or increased abrasion resistance of the dry ink.

Hence none of the prior art suggests the use of p-toluenesulfonamide for hydrophilic coatings with decreased friction or increased abrasion resistance.

### BRIEF DESCRIPTION OF THE INVENTION

The present invention thus relates to a device, suitably a medical device, having a substrate polymer surface carrying thereon a hydrophilic coating comprising cross-linked hydrophilic polymer and p-toluenesulfonamide.

The invention also relates to a method for the preparation of a device, suitably a medical device, having a substrate polymer surface carrying thereon a hydrophilic coating comprising cross-linked hydrophilic polymer and p-toluenesulfonamide, said method comprising the following steps:
(i) providing a device having a substrate polymer surface,
(ii) providing a coating solution comprising 0.1-20% by weight of a hydrophilic polymer which may be cross-linked, 0-5% by weight of additive(s), 0-40% by weight of plasticizers, 0.5-5% of p-toluenesulfonamide, and 50-99.4% of solvent(s)
(iii) applying said coating solution to said substrate polymer surface,
(iv) evaporating at least a part of the solvent(s) from said coating solution present on said substrate polymer surface, and curing said hydrophilic polymer.

The invention also relates to a device, suitably a medical device, having a substrate polymer surface carrying thereon a hydrophilic coating comprising cross-linked hydrophilic polymer and p-toluenesulfonamide obtainable by the above method and to coating solutions useful for the coating process.

### DETAILED DESCRIPTION OF THE INVENTION

The present invention is based on the finding that p-toluenesulfonamide provides advantageous properties to coatings comprising cross-linked hydrophilic polymers, such as lower friction when wet and higher abrasion resistance.

Basically, the coating according to the invention may be applied to any type of substrate. However, the coating according to the invention is particularly useful in the case of substrate polymer surfaces of polymers such as polyurethanes and copolymers thereof, or polyether block amides such as Pebax™ or other polymer materials including polyvinyl chloride, polyamide, silicone, styrene-ethylene/butylene-styrene block copolymers (SEBS), styrene-isoprene-styrene block copolymers (SIS), styrene-ethylene/propylene-styrene block copolymers (SEPS), ethylene-vinyl acetate copolymers (EVA), polyethylene (PE), metallocene-catalyzed polyethylene, and copolymers of ethylene and propylene or mixtures of such polymers.

For some combinations of substrate polymers and hydrophilic coatings, a primer coating may advantageously be applied before application of the coating solution. In some embodiments, the primer coating may be prepared from a diluted solution of the coating solution.

It is believed that highly plasticized polymeric materials like soft PVC will be less useful as substrates according to the invention as the fairly hydrophobic plasticizers for such materials tend to migrate into the coating. This reduces the wettability of the coating and interferes with the cross-linking reaction, especially when the drying period after the application of the polymer solution (e.g. dipping of the substrate polymer (the device)) is long. Hence the substrate polymer onto which the coating is applied is preferably non-plasticized. However, thin primer coatings of soft PVC contain too small an amount of hydrophobic plasticizer to interfere with the coating according to the invention and may in fact be quite useful in connection with certain substrates.

The surface on which the hydrophilic coating is applied may be the full surface of the substrate polymer surface or a part of the surface. In one embodiment, a part of the surface may be masked with a film or the like so as to form a predetermined pattern for the hydrophilic coating on the surface. Likewise, the substrate polymer surface on the device may cover the full surface of the device or a part thereof.

Typical examples of the hydrophilic polymer, which may be cross-linked are polyvinyl pyrrolidone, polyvinyl alcohol, poly(meth)acrylic acid, poly(meth)acrylic amides, polyethylene glycol, carboxymethylcellulose, cellulose acetate, cellulose acetate propionate, chitosan, polysaccharides, or any other hydrophilic homopolymer, or a copolymer of two or more of the monomers; N-vinyl pyrrolidone, vinyl alcohol, (meth)acrylic acid, (meth)acrylic amides, (meth)acrylic esters such as hydroxyethyl methacrylate, maleic anhydride, maleimide, methyl vinyl ether, alkyl vinyl ethers with vinylic side chains, and other unsaturated monomers. Furthermore, the hydrophilic polymer may be any blend of these homopolymers or copolymers. Other radiation curing hydrophilic polymers comprising unsaturated vinylic double bonds may also suitably be used for the coating. Such polymers may be achieved by copolymerising an acrylic substance like dimethylaminoethylmethacrylate with N-vinyl pyrrolidone, methacrylic acid, methacrylic esters, methyl vinyl ether etc. Such polymers are typically coated to the surface and ultimately radiation cured. A hydrophilic polymer useful for the coating may further be achieved by adding monomers of acrylic nature to the above-mentioned types of polymers. All the polymers can potentially be cross-linked by UV, electron beam or gamma irradiation.

Hydrophilic polymers containing active hydrogens capable of reacting with isocyanate groups may suitably be used in urethane type coatings. Such a coating is prepared by first coating an isocyanate compound onto the substrate polymer surface where such coating either adheres or covalently bonds to reactive groups at the surface. Secondly, a hydrophilic, reactive polymer is coated on top of such dried coating containing isocyanate groups. Said polymers may contain —OH, —SH, —NH—, —NH₂ and —CONH₂ groups. The polymers may be acrylic polymers and copolymers comprising acrylamide, hydroxyethyl acrylate, acrylic acid, polyethylene glycol methacrylate, polypropylene glycol methacrylate and the like. Furthermore, polyethylene glycols and polyvinyl pyrrolidone are useful for such hydrophilic coatings.

According to the invention, the hydrophilic polymer may be one particular type of hydrophilic polymer or it may be a blend of hydrophilic polymers, such as those listed above.

The hydrophilic polymer for the coating is preferably selected from the group of polyvinyl pyrrolidone or copolymers thereof, e.g. polyvinyl pyrrolidone-vinyl acetate copolymers. These types of hydrophilic polymers are very useful for cross-linking by radiation.

In one embodiment, the substrate polymer surface is polyurethane. In a further embodiment, the hydrophilic polymer which may be cross-linked is polyvinyl pyrrolidone. In particular, the substrate polymer surface is polyurethane and the hydrophilic polymer is polyvinyl pyrrolidone, such as PVP K-120.

When using the pure polyvinyl pyrrolidone (poly(N-vinyl-2-pyrrolidone, PVP), various chain lengths may be selected each giving various characteristics to the coating. Typically, such polyvinyl pyrrolidone polymers have a number average molecular weight of above 1×10⁶ g/mol. As an example, PVP K-120 with a molecular weight of 3.5×10⁶ g/mol can be selected, but other types of PVP with other molecular weights may also be used. In general, the higher the molecular weight of the PVP useful for the coating, the smaller the amount of PVP (w/w-%) that will give an abrasion-resistant, slippery surface of the wet coating, is needed. It is believed, that long PVP chains provide more points of intermingling with the substrate than short PVP chains (and hence good abrasion resistance and cross-linking) as well as larger domains of PVP far from the surface, which can bind water tightly and hence cause low friction and slow drying out.

For coating of the substrate polymer surface a coating solution is prepared. Suitably, the hydrophilic polymer(s), which may be cross-linked constitute(s) 0.1-20%, preferably 0.3-15%, more preferred 1-10%, or even more preferred 2-6% by weight of the coating solution.

Any solvent can in principle be used for the preparation of the coating solution of the invention. However, the solvent preferably comprises a volatile or fairly volatile solvent. The terms “volatile solvent” and “fairly volatile solvent” should be seen in the light of the evaporation rate. For this purpose, the evaporation rate relative to butyl acetate is typically used to provide a certain guideline in this respect (see in particular A. Saarnak, C. M. Hansen: “Löslighedsparametrar, Karaktärisering av färgbindemedel och polymerer”, publication from the Scandinavian Paint and Printing Ink Research Institute, Hørsholm, Denmark, May 1982 (in Swedish)). According to this paper, the evaporation rate (ER) is “Fast” if it is more than 3.0 times greater than that of butyl acetate (ER=1.0), i.e. ER>3.0; “Medium” if 0.8<ER<3.0; “Slow” if 0.1<ER<0.8; and “Very slow” if ER<0.1. “Volatile” and “Fairly volatile” correspond to a “fast” and “medium” evaporation rate, respectively. Volatile and fairly volatile solvents typically have a boiling point of up to 120° C.

Examples of volatile and fairly volatile solvents are acetone, 1,3-dioxolane, ethanol, ethyl acetate, methanol, methyl ethyl ketone (2-butanone), tetrahydrofuran (THF), isobutanol (2-methyl-1-propanol), butyl acetate, isobutyl acetate, methyl isobutyl ketone (4-methyl-2-pentanone), 1-propanol, and 2-propanol.

Especially preferred solvents include 1,3-dioxolane and other ethers, acetone and other ketones, dimethyl sulfoxide and other sulfoxides, dimethyl formamide and other amides, N-methyl-2-pyrrolidone and other lactams, ethanol and other alcohols, glycols, glycol ethers, glycol esters, other esters, amines, heterocyclic compounds, morpholine and derivatives thereof, alkylated urea derivatives, liquid nitriles, nitroalkanes, haloalkanes, haloarenes, trialkyl phosphates, dialkyl alkanephosphonates, and other commonly known organic solvents. The preferred solvents may either be used singly or in combination. Currently preferred solvents are selected from ethanol, N-methyl-2-pyrrolidone, dimethyl sulfoxide, acetone, 1,3-dioxolane and dimethyl formamide or mixtures thereof.

In a preferred embodiment, the coating solution comprises at least one of ethanol, acetone, dimethyl formamide and 1,3-dioxolane, and at least one of N-methyl-2-pyrrolidone and dimethyl sulfoxide. In a particular embodiment, the coating solution comprises 1) ethanol and N-methyl-2-pyrrolidone, or 2) ethanol and dimethyl sulfoxide, or 3) ethanol, N-methyl-2-pyrrolidone and dimethylsulfoxide. In another embodiment, the coating solution comprises 1) acetone and N-methyl-2-pyrrolidone, or 2) acetone and dimethyl sulfoxide, or 3) acetone, N-methyl-2-pyrrolidone and dimethylsulfoxide.

The most preferred solvent is ethanol, suitably in admixture with N-methyl-2-pyrrolidine (NMP).

Typically, the coating solution comprises comprises 50-99.4%, e.g. 60-98%, or more preferred 80-95%, by weight of solvent(s).

Suitably, the coating solution comprises 3-6% by weight of NMP and 80-95%, preferably 85-95% by weight of ethanol. More suitably, the hydrophilic polymer for cross-linking is polyvinyl pyrrolidone and the coating solution comprises 3-6% by weight of NMP and 80-95%, preferably 85-95% by weight of ethanol.

The coating solution and the coating of the invention may contain a plasticizer.

The preferred plasticizers are acetyl triethyl citrate, dimethyl sulfone, ethylene carbonate, glycerol diacetate, glycerol triacetate, hexamethylphosphoramide, isophorone, methyl salicylate, N-acetyl morpholine, propylene carbonate, quinoline, sulfolane, triethyl citrate, and triethyl phosphate. Particular examples are acetyl triethyl citrate, glycerol diacetate, glycerol triacetate, and triethyl citrate. The plasticizers may be used singly or in combination.

The plasticizer(s) may constitute(s) 0-40% by weight of the coating solution.

According to one embodiment of the invention the hydrophilic polymer which may be cross-linked is polyvinyl pyrrolidone and the coating solution does not contain a plasticizer as described above.

One or more additives may be included in the polymer solution, e.g. so as to facilitate the cross-linking of the hydrophilic polymer or so as to improve bonding of the polymer to the substrate surface. Such additives are known in the art and may include photoinitiators, e.g. as described in WO 98/58990. A suitable example of a photoinitiator is Esacure® KIP 150.

Furthermore, anti-infective agents could be included in the coating/coating solution if desired.

Thus, according to a further aspect, the present invention relates to a coating solution for the preparation of a cross-linked hydrophilic coating.

In one embodiment, the coating solution comprises:

0.1-20% by weight of hydrophilic polymer which may be cross-linked,

0-5% by weight of additive(s),

0-40% by weight of plasticizer(s),

0.5-5% by weight p-toluenesulfonamide, and

30-99.4%, preferably 50-99.4% by weight of solvent(s).

This is a fairly general recipe applicable for a wide range of substrates and hydrophilic polymers. One should, however, preferably take into account the recommendations given above with respect to the selection of a substrate, the hydrophilic polymer, the solvent(s), etc.

Thus, in a preferred embodiment, the polymer solution comprises:

2-10% by weight of polyvinyl pyrrolidone,

0-5% by weight of additive(s), preferably 0.03-0.5% Esacure KIP 150 photoinitiator

0.5-5%, by weight p-toluenesulfonamide,

0% plasticizer, and

80-97.5% by weight of a solvent selected from ethanol, N-methyl-2-pyrrolidone, dimethyl sulfoxide, acetone, 1,3-dioxolane and dimethyl formamide and mixtures of any of these solvents.

In a further embodiment, the polymer solution comprises:

2-6% by weight of polyvinyl pyrrolidone as the hydrophilic polymer,

0-5% by weight of additive(s), preferably 0.03-0.5% Esacure KIP 150 photoinitiator

0.5-3% by weight p-toluenesulfonamide,

0% plasticizer, and

86-97.5% by weight of a solvent selected from ethanol, N-methyl-2-pyrrolidone, dimethyl sulfoxide, acetone, 1,3-dioxolane and dimethyl formamide and mixtures of any of these solvents.

The invention also provides a method for the applying a cross-linked hydrophilic coating of a hydrophilic polymer on a substrate polymer surface of a device, suitably a medical device.

In more detail, the method comprises the following steps (i)-(iv).

Step (i)

The substrate polymer surface may be the native surface of a device, suitably a medical device, or may be surface treated so as to facilitate strong bonding of the hydrophilic coating to the substrate polymer. The surface of the substrate polymer may be the complete physical surface or a fraction thereof. For many medical devices, it is only necessary to coat the part of the substrate polymer surface that comes into direct contact with the surface of living tissue when in use. The step of providing a substrate polymer having the substrate polymer surface will be evident for the person skilled in the art.

Step (ii)

The composition of the coating solution is important for the method of the invention. The amount of hydrophilic polymer, solvent, p-toluenesulfonamide, plasticizer(s) and additives are described above. The solution may be prepared by mixing the components to obtain the coating solution. The mixing order is not particularly critical as long as a homogeneous (and possibly clear) solution is obtained. Thus, the step of actual preparation of the coating solution will be evident for the person skilled in the art in view of the above directions with respect to choice of components.

Step (iii)

Application of the coating solution to said substrate polymer surface is conducted following conventional methods such as dip coating, spray coating, application by means of brushes, rollers, etc., as will be evident for the person skilled in the art. With due consideration of the production process, it is preferred that the application of the coating solution to the substrate polymer surface is performed by dipping the device, suitably a medical device (or the relevant surface thereof) into the coating solution.

In a preferred embodiment, the coating solution is applied to the substrate polymer surface in one single application step, such as in a one-dip process.

In another preferred embodiment, the coating solution is applied to the substrate polymer surface in two or three individual application steps, in particular in two individual application steps, such as in a two-dip process.

The dipping process typically takes place by immersing the polymer substrate in the coating solution and then withdrawing it at a speed of 0.2-20 cm per second at a temperature in the range of 0-100° C., or at a speed of 1-15 cm per second at room temperature.

For all embodiments, it should be understood that the substrate polymer may be primed in one or more preceding step(s) and that such (a) preceding step(s) may be performed in addition to the before-mentioned application step(s) (e.g. one-dip process or two-dip process) of applying the coating solution. As mentioned above, the primer coat may be formed from a dilute solution of the coating solution.

Hence, in one embodiment, the application of the coating solution (one or two dips, in particular one dip) to the substrate polymer surface (step (iii) is preceded by a priming step in which a dilute solution of the coating solution (e.g. using a dilution factor of 1-8, and typically diluted with a solvent or a solvent mixture as above, most typically ethanol) is applied to the polymer substrate surface in one or more steps (in particular in one step). In particular, both application steps (the priming step and step (iii)) involve dipping of the substrate polymer surface in the primer solution and coating solution, respectively. More preferred, the priming step and step (iii) are each performed by one dip of the substrate polymer surface (or the relevant part thereof) into the relevant solution (i.e. the primer solution and the coating solution, respectively).

Step (iv)

After application of the coating solution to the substrate polymer surface (e.g. a primer surface), any solvent or at least a part thereof is evaporated from the coating solution present on said substrate polymer surface. The aim is to remove the most volatile components. The volatile components may be removed by passive evaporation, by leading a stream of air over the surface of the substrate polymer, or by applying a reduced pressure over the surface of the substrate polymer. The drying typically takes place at a temperature in the range of 20-150° C. for 1-60 minutes, such as at 50-120° C. for 5-45 minutes. It may be necessary or desirable to increase the temperature of the substrate polymer or the air surrounding the substrate polymer to speed up the evaporation process. Preferably, the evaporation process is facilitated by drying the substrate polymer with the coating solution at a temperature in the range of 25-100° C. depending on the thermostability of the substrate polymer. Typically, the substrate polymer (e.g. a medical device) is dried in an oven.

Although the curing of the hydrophilic polymer of the coating solution may be effected or at least initiated upon the at least partial evaporation of the solvent, it is often desirable to specifically induce curing (cross-linking) of the hydrophilic polymer. Most advantageously, the free-radical curing (and cross-linking) is performed by application of radiation, e.g. UV-irradiation. The method of curing, in particular the frequency of the UV light, depends on the choice of photoinitiator. The person skilled in the art will know the means and procedures necessary for efficient curing and to obtain the desired degree of cross-linking, see e.g. “Radiation Curing in Polymer Science and Technology”, volumes. I-IV, eds. J. P. Fouassier and J. F. Rabek, Elsevier, London, 1993.

In the present context, the terms “cross-linked” and “cured” when referring to a polymer or polymers are intended to mean attachment of two chains of polymer molecules by covalent chemical bonds, possibly through linker(s). “Cross-linked” and “cured” also means such covalent chemical bonds occuring between chains of similar nature.

In a preferred embodiment of the above method, the hydrophilic coating is prepared by dipping a device, suitably a medical device having a substrate polymer surface of polyurethane in a solution of the preferred hydrophilic polymer (i.e. polyvinyl pyrrolidone), a photoinitiator (such as Esacure® KIP 150), p-toluenesulfonamide and one or more solvents selected from ethanol, N-methyl-2-pyrrolidone, dimethyl sulfoxide, acetone, 1,3-dioxolane and dimethyl formamide. The device is subsequently dried in an oven at a temperature of 25-100° C., typically for 5-60 minutes, so as to remove a substantial portion of the solvent and irradiated with specific ultraviolet light to effect cross-linking.

The present invention also provides a device, suitably a medical device, comprising a substrate polymer surface having thereon a hydrophilic coating of a cross linked hydrophilic polymer, said medical device being obtainable by the method described above.

The invention is further illustrated by means of the following examples.

Experimentals

All quantities indicated herein as “percentages” refer to percentages by weight.

Materials

PVP K-120 (molecular weight 3.5×10⁶ g/mol) was obtained from ISP.

NMP (N-methylpyrrolidone) was from Riedel-de Haën

Toluene-4-sulfonamide (p-toluenesulfonamide) was from Fluka

99.5% ethanol was from Bie & Berntsen

TMPTMA (trimethylolpropane trimethacrylate) was from Bisomer

Esacure® KIP 150 was obtained from Lamberti SpA.

### Examples 1-6

Preparation of Coatings with p-toluenesulfonamide

The ingredients for the coatings solution are given in table 1 below. The liquids were mixed, and the solids were added over a period of about 20 minutes so that no lumps of PVP were formed during magnetic stirring. Stirring was continued for at least 30 minutes to ensure perfect dissolution of the solids. Polyurethane-coated stainless steel or nitinol guide wires were dipped in the solution and withdrawn at a speed of approximately 5.5 m/min (92 mm/s). The guidewires were dried for 26 minutes at 90° C. and UV cured.

After swelling in water the following subjective tests were performed:

Friction on a scale from 0 to 5, where 0 was extremely slippery and 5 was not slippery at all.

Soft abrasion loss of the wet guidewires on a scale from 0 to 5 with half-integer steps, where 0 corresponded to no abrasion loss by running two fingers down the length of the guide wire, and 5 corresponded to total loss of the coating.

Hard abrasion loss of the wet guidewires on a scale from 0 to 5, where 0 corresponded to no abrasion loss by pulling the guidewire out through an introducer tip at an angle, and 5 corresponded to total loss of the coating.

The result of the test is shown in Table 1.

TABLE 1
Performance of coatings with and without p-toluenesulfonamide.

| | % | | % Esacure | | % | | | | Soft | Hard |
|---|---|---|---|---|---|---|---|---|---|---|
| Example | PVP | % | KIP | | TMP- | | % | | abrasion | abrasion |
| no. | K-120 | NMP | 150 | % benzophenone | TMA | % p-toluenesulfonamide | ethanol | Friction | loss | loss |
| 1 | 3 | 5 | 0.3 | | | 1.2 | 90.5 | 0.5 | 1 | 0 |
| 2 | 3 | 5 | 0.3 | | | 0.6 | 91.1 | 2 | 0 | 1 |
| 3 | 3 | 5 | 0.3 | | | | 91.7 | 5 | 0 | 1 |
| 4 | 3 | 5 | 0.06 | | | | 91.94 | 5 | 3 | 0 |
| 5 | 3 | 5 | 0.06 | | 0.12 | | 91.82 | 2 | 2 | 3 |
| 6 | 3 | 5 | | 0.06 | 0.12 | | 91.82 | 5 | 3 | 0 |

A very low friction and low soft and hard abrasion loss resulted when a guidewire was coated with PVP containing 1.2% p-toluenesulfonamide (example 1). When only 0.6% p-toluenesulfonamide was added (example 2), a somewhat higher friction resulted than in example 1, whereas the soft and hard abrasion losses were still small. By contrast, the corresponding coating without p-toluenesulfonamide (example 3) had very high friction, although the soft and hard abrasion losses remained low. In example 4 an attempts was made to reduce the friction by adding less photoinitiator and hence reducing the amount of PVP crosslinking, but the friction was not reduced and the soft abrasion loss actually increased. Example 5 shows that addition of 0.12% TMPTMA did reduce the friction to the level of example 2 (with 0.6% p-toluenesulfonamide), but at the same time the soft and hard abrasion losses increased considerably. In example 6 substitution of Esacure KIP 150 with benzophenone (a strictly hydrogen-abstracting photoinitiator) again gave a very high friction and so was unsuccessful. In conclusion the addition of p-toluenesulfonamide to the coating solution was necessary in order to obtain low values of friction, soft abrasion loss, and hard abrasion loss.

1. A device having a substrate polymer surface carrying on a least a part of the substrate polymer surface a hydrophilic coating comprising a cross-linked hydrophilic polymer and p-toluenesulfonamide.

2. The device according to claim 1 wherein the substrate polymer surface is polyurethane.

3. The device according to claim 1 wherein the hydrophilic polymer is polyvinyl pyrrolidone.

4. The device according to claim 3 wherein the hydrophilic polymer is PVP K-120.

5. A method for the preparation of a device having a substrate polymer surface carrying on a least a part of the substrate polymer surface a hydrophilic coating comprising a cross-linked hydrophilic polymer and p-toluene-sulfonamide, said method comprising the following steps:
(i) providing a device having a substrate polymer surface,
(ii) providing a coating solution comprising 0.1-20% by weight of a hydrophilic polymer which may be cross-linked, 0-5% by weight of additive(s), 0-40% by weight of plasticizers, 0.5-5% of p-toluenesulfonamide, and 50-99.4% of solvent(s),
(iii) applying said coating solution to said substrate polymer surface,
(iv) evaporating at least a part of the solvent(s) from said coating solution present on said substrate polymer surface, and curing said hydrophilic polymer.

6. A method for the preparation of a device according to claim 5 wherein the coating solution comprises 2-10% by weight of polyvinyl pyrrolidone as the hydrophilic polymer, 0-5% by weight of additive(s), 0.5-5% by weight p-toluenesulfonamide, and
80-97.5% by weight of solvents selected from ethanol, N-methyl-2-pyrrolidone, dimethyl sulfoxide, acetone, 1,3-dioxolane and dimethyl formamide, or a mixture of any of these solvents.

7. The method according to claim 5, wherein the coating solution is applied to said substrate polymer surface in one single application step.

8. The method according to claim 5, wherein the substrate polymer surface is polyurethane.

9. The method according to claim 5, wherein the hydrophilic polymer is polyvinyl pyrrolidone.

10. The method according to claim 9 wherein the hydrophilic polymer is PVP K-120.

11. The method according to claim 5, wherein the coating solution comprises a mixture of ethanol and N-methyl-2-pyrrolidone as the solvent.

12. A device having a substrate polymer surface carrying on a least a part of the substrate polymer surface a hydrophilic coating comprising a cross-linked hydrophilic polymer and p-toluenesulfonamide, said device being obtainable by the method of claim 5.

13. The device according to claim 1 wherein the device is a medical device.

14. The method according to claim 5 wherein the device is a medical device.

15. A coating solution comprising 0.1-20% by weight of a hydrophilic polymer which may be cross-linked, 0-5% by weight of additive(s), and 0.5-5% of p-toluenesulfonamide, 0-40% by weight of plasticizers and 50-99.4% by weight of solvent(s).

16. A coating solution according to claim 15 comprising 2-10% by weight of polyvinyl pyrrolidone as the hydrophilic polymer, 0-5% by weight of additive(s), 0.5-5% by weight p-toluenesulfonamide, and 80-97.5% by weight of solvents selected from ethanol, N-methyl-2-pyrrolidone, dimethyl sulfoxide, acetone, 1,3-dioxolane and dimethyl formamide, or a mixture of any of these solvents.

17. A coating solution according to claim 15 wherein the hydrophilic polymer is polyvinyl pyrrolidone and the coating solution comprises 3-6% by weight of NMP and 85-95% by weight of ethanol as the solvent.
